(19) United States
(12) Patent Application Publication
(10) Pub. No.: US 2015/0167969 A1
(43) Pub. Date: Jun. 18, 2015

(54) PROCESSES UTILIZING FERMENTATION VINASSE FOR PRODUCING ENERGY-DENSE BIOMASS AND BIOMASS SUGARS

(71) Applicant: API Intellectual Property Holdings, LLC, Atlanta, GA (US)

(72) Inventors: Theodora RETSINA, Atlanta, GA (US); Vesa PYLKKANEN, Atlanta, GA (US); Ryan O'CONNOR, Minnetrista, MN (US)

(21) Appl. No.: 14/633,326

(22) Filed: Feb. 27, 2015

Related U.S. Application Data
(63) Continuation-in-part of application No. 13/874,761, filed on May 1, 2013.
(60) Provisional application No. 61/947,212, filed on Mar. 3, 2014.
(60) Provisional application No. 61/641,435, filed on May 2, 2012.

(51) Int. Cl. F23G 7/10; C12P 3/00; F23G 5/04; C10J 3/02; F23G 5/033; C12P 7/10; C12P 7/16

(52) U.S. Cl. CPC F23G 7/10; C12P 7/10; C12P 3/00; C12P 7/16; C10J 3/02; F23G 5/033; F23G 5/04; C10J 2300/1625; C10J 2300/0909; C10J 2300/0916

(57) ABSTRACT

This invention provides a process for producing biomass pellets and fermentable sugars from cellulosic biomass, comprising: extracting cellulosic biomass feedstock with steam and/or hot water to produce an extract liquor containing hemicellulosic oligomers, dissolved lignin, and cellulose-rich solids, wherein at least some of the steam and/or hot water is derived from an internal or external source of fermentation vinasse; separating at least a portion of the hemicellulosic oligomers from the cellulose-rich solids, to produce intermediate solids; hydrotorrefying the intermediate solids to produce the energy-dense biomass, wherein water for hydrotorrefaction is derived, at least in part, from the internal or external source of fermentation vinasse; pelletizing the energy-dense biomass to form biomass pellets; and hydrolyzing the hemicellulosic oligomers into fermentable sugars. The fermentation vinasse may be recycled from downstream operations involving fermentation of the sugars. Or the fermentation vinasse may be obtained from a first-generation ethanol plant, for example.

### PRIORITY DATA

This patent application is a non-provisional application claiming priority to U.S. Provisional Patent App. No. 61/947,212, filed Mar. 3, 2014, and further is a continuation-in-part of U.S. patent application Ser. No. 13/874,761, filed May 1, 2013, which in turn claims priority to U.S. Provisional Patent App. No. 61/641,435, filed May 2, 2012.

### FIELD OF THE INVENTION

The present invention generally relates to processes for preparing energy-dense biomass for combustion, while also recovering fermentable sugars from the biomass.

### BACKGROUND OF THE INVENTION

Wood and biomass burning is making a comeback after over century of domination by coal, petroleum, and natural gas for power generation. The availability of energy-dense fossil fuels and efficient transportation networks made centralized power production the technology of choice. In the 21st century, biomass heat and power plants and district heating are enjoying a renaissance. This popularity is driven in part by the carbon-neutral nature of most biomass (i.e., no net CO₂ emissions). The rising cost of fossil fuels and incentives for switching drive consumer decisions toward renewable energy. Also, renewable-energy portfolio mandates require that utilities construct renewable power plants.

One challenge to combusting biomass is its high moisture content. Living and freshly cut biomass typically contains moisture between 40% and 60%. In loose storage, the biomass dryness can reach air-dry moisture of about 10%. This drying of wood is slow, typically requiring at least a full summer season. This necessitates double handling and increases procurement cost. It can be advantageous to first pelletize biomass, which can drive moisture out of the biomass, by using part of the biomass energy, waste heat, or a fossil fuel. The final moisture from pelletizing is typically 5-7%, which is similar to moisture of coal. Boiler efficiencies increase approximately half a percent with each percentage removal of moisture.

In biomass, cellulose and hemicellulose each have about half of the calorific heat value of coal, because of high oxygen content of polymeric sugar constituents. Lignin has a similar calorific heat value to coal, but sulfur is nearly absent. The combined energy content of biomass is typically 8,000-9,000 Btu/lb, as compared to 10,000-14,000 Btu/lb in coal. Because of high oxygen content and moisture in biomass, the boiler efficiency for biomass firing typically ranges from 50-65%. A large portion of heat generated in combustion escapes as steam through the stack. Therefore, converting coal-burning boilers to biomass firing may reduce boiler capacity by as much as 60%. There is a need to maximize utilization of these assets, and therefore more energy-dense biomass is desired.

Feeding irregularly shaped biomass also represents a challenge. Pelletizing can produce uniformly sized material that does not bridge or lodge easily in a storage silo. On the other hand, the pelletized material can absorb moisture, if stored loosely outdoors.

Another obstacle is presented by the ash in the biomass. Ash content of biomass typically varies between 0.4% and 15%. Hardwood and softwood stem and forest trimmings contain only 0.4% to 0.8% ash that is rich in calcium and potassium. Other biomass materials including pulp and paper sludge, paper waste, recycled paper and construction waste, can contain up to 30% ash. Such ash includes minerals in plant capillaries, dirt on the surface, and coating in the paper. The wood exposed to salt water contains elevated levels of sodium and chlorides.

Agricultural residues of annual plants, such as corn stover, corn fiber, wheat straw, sugarcane bagasse, rice straw, oat straw, barley straw, and miscanthus can contain up to 10% or more ash that is rich in silica, potassium, and chlorine. The agricultural residue material is very lean in sulfur, typically less than 0.1%, versus coal sulfur content of 0.5-7.5%. Significant minerals in these annual agricultural residues include potassium, sodium, silica, calcium, and corrosive halogens such as chlorides.

Upon combustion at high temperatures, metals and halogens volatilize to aerosols and carry over from the boiler with flue gas. The cooling of fly ash creates microscopic particles that are found to cause respiratory illnesses. Flue-gas treatment for particulate removal includes cyclones, scrubbers, and electrostatic precipitators (ESP). These environmental controls in the central power plant are expensive and, in domestic applications, tend to be cost-prohibitive. Recent Maximum Achievable Control Technology (MACT) legislation by the U.S. EPA seeks to control particulate emissions from large biomass power plants. Other minerals such as calcium and silica remain in the bottom of the boiler and have tendency to form clinkers and to scale (slag) in the boiler tubes. Alkaline chloride salts can cause corrosion of the boiler tubes.

What are needed are processes and apparatus to prepare biomass, including wood and agricultural residues, into clean, energy-dense biomass for improved combustion, with or without pelletizing the biomass. The energy-dense biomass should be capable of being fired alone or in combination with another solid fuel. It would be desirable for these processes to also have good potential to recover various co-products, such as sugars, sugar fermentation products, furfural, levulinic acid, fertilizers, and lignin.

### SUMMARY OF THE INVENTION

The present invention addresses the aforementioned needs in the art.

Some variations provide a process for producing energy-dense biomass and fermentable sugars from cellulosic biomass, the process comprising:

(a) providing a feedstock comprising cellulosic biomass;

(b) extracting the feedstock with steam and/or hot water under effective extraction conditions to produce an extract liquor containing hemicellulosic oligomers, dissolved lignin, and cellulose-rich solids, wherein at least some of the steam and/or hot water is derived from an internal or external source of fermentation vinasse;

(c) separating at least a portion of the hemicellulosic oligomers from the cellulose-rich solids, to produce intermediate solids;

(d) torrefying the intermediate solids to produce the energy-dense biomass;

(e) hydrolyzing the hemicellulosic oligomers into fermentable sugars; and

(f) recovering the fermentable sugars.

In some embodiments, step (d) utilizes hydrotorrefaction, i.e. torrefaction in the presence of water. In some embodiments, the extraction solution comprises pressurized hot water, which may be used also for hydrotorrefaction when employed. The extraction solution may further contains sulfur dioxide, sulfurous acid, sulfuric acid, or any combination thereof

In some embodiments, step (c) includes washing the cellulose-rich solids using an aqueous wash solution, to produce a wash filtrate; and optionally combining at least some of the wash filtrate with the extract liquor. In certain embodiments, step (c) includes pressing the cellulose-rich solids to produce dewatered cellulose-rich solids and a press filtrate; and optionally combining at least some of the press filtrate with the extract liquor.

The process may further comprise refining or milling the intermediate cellulose-rich solids prior to step (d). The process may further comprise combusting the energy-dense biomass to produce power and/or heat. Alternatively, or additionally, the process may further comprise gasifying the energy-dense biomass to produce syngas.

The process further comprises, in some embodiments, a step of fermenting the fermentable sugars to a fermentation product. Optionally, the step of fermenting the fermentable sugars to a fermentation product generates fermentation vinasse that is recycled (and reused), at least in part, to step (b) (and/or to step (d) when hydrotorrefaction is employed). Alternatively, or additionally, the fermentation vinasse may be obtained from a separate, first-generation ethanol plant, directly or indirectly, for example.

In some embodiments, the process further comprises converting at least a portion of the hemicelluloses into furfural. At least a portion of the furfural may degrade to char, which may then be recovered with the energy-dense biomass. Similarly, the process may co-produce 5-hydroxymethylfurfural and/or levulinic acid from part of the cellulose-rich solids or the intermediate solids.

In some embodiments, the process further comprises pelletizing the intermediate solids and/or the energy-dense biomass, to produce biomass pellets.

Other variations of the invention provide a process for producing biomass pellets and fermentable sugars from cellulosic biomass, the process comprising:

(a) providing a feedstock comprising cellulosic biomass;

(b) extracting the feedstock with steam and/or hot water under effective extraction conditions to produce an extract liquor containing hemicellulosic oligomers, dissolved lignin, and cellulose-rich solids;

(c) separating at least a portion of the hemicellulosic oligomers from the cellulose-rich solids, to produce intermediate solids;

(d) hydrotorrefying the intermediate solids to produce the energy-dense biomass, wherein water for hydrotorrefaction is derived, at least in part, from an internal or external source of fermentation vinasse;

(e) pelletizing the energy-dense biomass to form the biomass pellets;

(f) hydrolyzing the hemicellulosic oligomers into fermentable sugars; and

(g) recovering the fermentable sugars.

In some embodiments, the extraction solution comprises pressurized hot water. The extraction solution may further contain sulfur dioxide, sulfurous acid, sulfuric acid, or any combination thereof.

The process may further comprise refining or milling the intermediate cellulose-rich solids prior to step (d).

The process may include combusting the biomass pellets to produce power and/or heat. In some embodiments, the biomass pellets have an energy content from about 8,500 Btu/lb to about 12,000 Btu/lb on a dry basis. The biomass pellets may also be gasified to produce syngas, which may be used for combined heat and power or for conversion to liquid fuels (e.g., Fischer-Tropsch synthesis to diesel fuels), for example.

In some embodiments, the process further comprises converting at least a portion of the hemicelluloses into furfural. Some or all of the furfural may degrades to char, and the char is optionally recovered with the energy-dense biomass. The process may also co-produce 5-hydroxymethylfurfural and/or levulinic acid from part of the cellulose-rich solids or the intermediate solids.

Some embodiments further comprise a step of fermenting the fermentable sugars to a fermentation product. This step may generate fermentation vinasse that is recycled, at least in part, to step (d) and/or to step (b). Alternatively, or additionally, fermentation vinasse is obtained from a separate, first-generation ethanol plant, and utilized in step (d) and/or step (b).

Certain variations provide a process for producing biomass pellets and fermentable sugars from cellulosic biomass, the process comprising:

(a) providing a feedstock comprising cellulosic biomass;

(b) extracting the feedstock with steam and/or hot water under effective extraction conditions to produce an extract liquor containing hemicellulosic oligomers, dissolved lignin, and cellulose-rich solids, wherein at least some of the steam and/or hot water is derived from an internal or external source of fermentation vinasse;

(c) separating at least a portion of the hemicellulosic oligomers from the cellulose-rich solids, to produce intermediate solids;

(d) hydrotorrefying the intermediate solids to produce the energy-dense biomass, wherein water for hydrotorrefaction is derived, at least in part, from the internal or external source of fermentation vinasse;

(e) pelletizing the energy-dense biomass to form the biomass pellets;

(f) hydrolyzing the hemicellulosic oligomers into fermentable sugars; and

(g) recovering the fermentable sugars.

### BRIEF DESCRIPTION OF THE FIGURE

FIG. 1 is a simplified block-flow diagram depicting the process of some embodiments of the present invention. Fermentation stillage or vinasse may be employed for any step(s) requiring water. Dashed lines indicate optional streams.

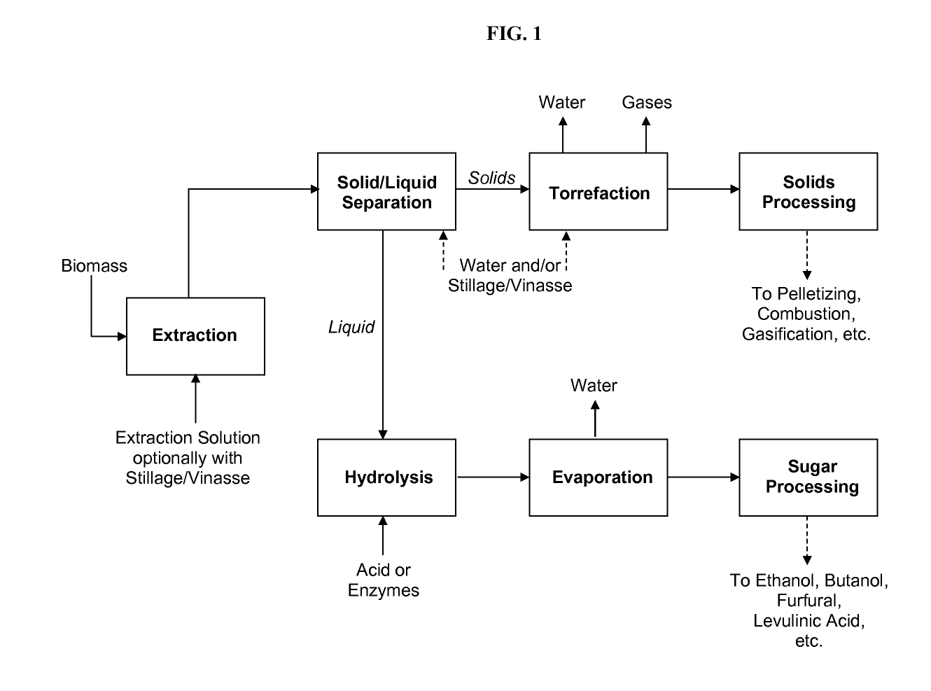

### DETAILED DESCRIPTION OF EMBODIMENTS OF THE INVENTION

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

The present invention is premised, at least in part, on the realization that pretreatment of biomass may be utilized to remove hemicellulose and cellulose from the biomass, and thereby significantly increase the energy density of the biomass. The pretreated biomass will also be cleaned of ash components, to reduce particulate emissions upon combustion of the biomass. The extract may be further treated to make fermentable sugars, and optionally fermentation products. In an integrated process, unused solids or other combustible components recovered at any point may be co-combusted with the pretreated biomass, or separately recovered.

Certain exemplary embodiments of the invention will now be described. These embodiments are not intended to limit the scope of the invention as claimed. The order of steps may be varied, some steps may be omitted, and/or other steps may be added. Reference herein to first step, second step, etc. is for illustration purposes only.

“Biomass,” for purposes of this disclosure, shall be construed as any biogenic feedstock or mixture of a biogenic and non-biogenic feedstock. Elementally, biomass includes at least carbon, hydrogen, and oxygen. The methods and apparatus of the invention can accommodate a wide range of feedstocks of various types, sizes, and moisture contents.

Biomass includes, for example, plant and plant-derived material, vegetation, agricultural waste, forestry waste, wood waste, paper waste, animal-derived waste, poultry-derived waste, and municipal solid waste. In various embodiments of the invention utilizing biomass, the biomass feedstock may include one or more materials selected from: softwood chips, hardwood chips, timber harvesting residues, tree branches, tree stumps, knots, leaves, bark, sawdust, off-spec paper pulp, cellulose, corn, corn stover, wheat straw, rice straw, sugarcane, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, carbohydrates, plastic, and cloth.

Selection of a particular feedstock or feedstocks is not regarded as technically critical, but is carried out in a manner that tends to favor an economical process. Typically, regardless of the feedstocks chosen, there can be (in some embodiments) screening to remove undesirable materials. The feedstock can optionally be dried prior to processing.

The feedstock employed may be provided or processed into a wide variety of particle sizes or shapes. For example, the feed material may be a fine powder, or a mixture of fine and coarse particles. The feed material may be in the form of large pieces of material, such as wood chips or other forms of wood (e.g., round, cylindrical, square, etc.). In some embodiments, the feed material comprises pellets or other agglomerated forms of particles that have been pressed together or otherwise bound, such as with a binder. The binder may be a lignin derivative, a sugar-degradation product (e.g., furfural), or a component released from the starting biomass, such as acetic acid or an acetate salt thereof.

All references to “water” herein include not only pure or substantially pure water, but any form of recycled water, process condensate, fermentor still bottoms (stillage), fermentation vinasse (such as from sugarcane ethanol processing), wastewater, and so on.

In some embodiments, the process starts as biomass is received or reduced to approximately ¼″ thickness. In a first step of the process, the biomass chips are fed to a pressurized extraction vessel operating continuously or in batch mode. The chips may be steamed or water-washed to remove dirt and entrained air. The chips are immersed with aqueous liquor or saturated vapor and heated to a temperature between about 100° C. to about 250° C., for example 150° C., 160° C., 170° C., 180° C., 190° C., 200° C., or 210° C. Preferably, the chips are heated to about 180° C. to 210° C. The pressure in the pressurized vessel may be adjusted to maintain the aqueous liquor as a liquid, a vapor, or a combination thereof. Exemplary pressures are about 1 atm to about 30 atm, such as about 3 atm, 5 atm, 10 atm, or 15 atm.

The aqueous liquor may contain acidifying compounds, such as (but not limited to) sulfuric acid, sulfurous acid, sulfur dioxide, acetic acid, formic acid, or oxalic acid, or combinations thereof. The dilute acid concentration can range from 0.01% to 10% as necessary to improve solubility of particular minerals, such as potassium, sodium, or silica. Preferably, the acid concentration is selected from about 0.01% to 4%, such as 0.1%, 0.5%, 1%, 1.5%, 2%, 2.5%, 3%, or 3.5%.

A second step may include depressurization of the extracted chips. The vapor can be used for heating the incoming woodchips or cooking liquor, directly or indirectly. The volatilized organic acids (e.g., acetic acid), which are generated or included in the cooking step, may be recycled back to the cooking.

A third step may include washing the extracted chips. The washing may be accomplished with water, recycled condensates, recycled permeate, or combination thereof. A liquid biomass extract is produced. A countercurrent configuration may be used to maximize the biomass extract concentration. Washing typically removes most of the dissolved material, including hemicelluloses and minerals. The final consistency of the dewatered cellulose-rich solids may be increased to 30% or more, preferably to 50% or more, using a mechanical pressing device.

The third step, or an additional step prior to drying (below), may include further hydrolyzing the extracted chips with enzymes or an acid to extract some of the cellulose as fermentable glucose. The removal of cellulose increases the heating value of the remaining lignin-rich solids. In certain embodiments, the heating value of the remaining solids can approach that of lignin, i.e. in the range of about 10,000 to 12,000 Btu/lb. In some preferred embodiments, the additional hydrolysis is mild hydrolysis that leaves a substantial portion of cellulose in the extracted solids. The mild hydrolysis can take advantage of the initial extraction (first step) of most or all of the hemicellulosic material, leaving a somewhat hollow structure. The hollow structure can increase the effectiveness of cellulose hydrolysis, such as by reducing mass-transfer limitations of enzymes or acids in solution.

When enzymes are employed for the cellulose hydrolysis, the enzymes are preferably cellulase enzymes. Enzymes may be introduced to the extracted chips along with the wash solution, e.g. water, recycled condensates, recycled permeate, or combinations thereof. Alternatively, or additionally, enzymatic hydrolysis may be carried out following washing and removal of hemicelluloses, minerals, and other soluble material.

Enzymes may be added to the extracted chips before or after mechanical pressing. That is, enzymatic hydrolysis may be carried out and then the solids pressed to final consistency; or, the solids may be pressed to high consistency (e.g., 30% or more) and then enzymes introduced to carry out cellulose hydrolysis. It may be beneficial to conduct refining or milling of the dewatered cellulose-rich solids prior to the enzymatic hydrolysis.

The enzymatic hydrolysis may be achieved in a separate unit, such as between washing and drying, or as an integrated part of washing. In some embodiments, at least a portion of enzymes are recycled in a batch or continuous process.

When an acid is employed for the cellulose hydrolysis, the acid may be selected from sulfuric acid, sulfurous acid, sulfur dioxide, formic acid, acetic acid, oxalic acid, or combinations thereof. Dilute-acid hydrolysis is preferred, to avoid sugar degradation. In some embodiments, sulfur dioxide is preferred for so enable better downstream acid recovery. Acids may be introduced to the extracted chips along with the wash solution, e.g. water, recycled condensates, recycled permeate, or combinations thereof. Alternatively, or additionally, acid hydrolysis may be carried out following washing and removal of hemicelluloses, minerals, and other soluble material.

Acids may be added to the extracted chips before or after mechanical pressing. That is, acid hydrolysis may be carried out and then the solids pressed to final consistency; or, the solids may be pressed to high consistency (e.g., 30% or more) and then acids introduced to carry out cellulose hydrolysis. It may be beneficial to conduct refining or milling of the dewatered cellulose-rich solids prior to the acid hydrolysis.

The acid hydrolysis may be achieved in a separate unit, such as between washing and drying, or as an integrated part of washing. In some embodiments, at least a portion of the acid is recycled in a batch or continuous process.

A fourth step may include drying of the extracted solids to a desired final moisture. The heat necessary for drying may be derived from combusting part of the starting biomass. Alternatively, or additionally, the heat for drying may be provided by other means, such as a natural gas boiler or other auxiliary fossil fuel, or from a waste heat source.

A fifth step may include preparing the biomass for combustion. This step may include refining, milling, fluidizing, compacting, torrefying, carbonizing, and/or pelletizing the extracted biomass. The biomass may be fed to a boiler in the form of fine powder, loose fiber, pellets, briquettes, extrudates, or any other suitable form. In some embodiments, pellets of extracted biomass (“biomass pellets”) are preferred. Using known equipment, biomass may be extruded through a pressurized chamber to form uniformly sized pellets or briquettes. Mild refining using a blow unit may be employed to disrupt the fibers and reduce particle size, since it may be beneficial to avoid longer fibers in pellets.

The energy-dense biomass will generally have higher energy density compared to a process that does not extract hemicellulosic sugars from the feedstock prior to combustion. Depleting the biomass of both hemicellulose and cellulose enriches the remaining material in lignin, which has a higher energy density than hemicellulose or cellulose.

In some embodiments, the extracted solids are fed to a torrefaction unit. Torrefaction is a form of mild pyrolysis at temperatures typically ranging between 200° C. to 325° C. During torrefaction, biomass properties are changed to obtain better fuel quality for combustion and gasification applications.

Torrefaction of biomass particles is well-known and is a process in which biomass particles are heated in a low-oxygen or oxygen-free environment. Volatile compounds within the particles are released, including water, and the cellular structure of the particles is degraded, resulting in a partial loss of mass and an increase in friability. Friability means the ability of a solid substance to be reduced to smaller pieces. Torrefaction also can enhance the moisture resistance of the solids. Torrefied particles have an enhanced energy value when measured in terms of heat energy per unit of weight. Torrefaction of biomass also can improve the grindability. This leads to more efficient co-firing in existing coal-fired power stations or entrained-flow gasification for the production of chemicals and transportation fuels.

The degree of torrefaction of biomass particles depends on several factors, including the level of heat applied, the length of time the heat is applied, and surrounding gas conditions (particularly with respect to oxygen level). Known biomass-torrefaction systems control the variables of heat, residence time, and oxygen levels to achieve consistent torrefied particles, typically employing mechanical means to convey the particles, such as rotating trays or screws.

In some embodiments, the energy density of the biomass pellet is from about 8,500 Btu/lb to about 12,000 Btu/lb on a dry basis, such as at least 9,000 Btu/lb or at least 10,000 Btu/lb on a dry basis.

A sixth step is combustion of the biomass, which in some embodiments is in the form of biomass pellets (e.g. following torrefaction). The biomass pellets are fed to a boiler and combusted, preferably with excess air, using well-known combustion apparatus. Boiler bottom may be fixed, moving, or fluidized for the best efficiency. The flue gas is cooled and fly ash is collected into gravity collectors.

The energy-dense biomass has lower inorganic emissions potential compared to the original cellulosic biomass, in preferred embodiments. The reason is that the energy-dense biomass will contain lower ash content compared to a process that does not extract inorganic components from the feedstock prior to combustion, in the manner disclosed herein. In some embodiments, the extracted biomass is sufficiently low in ash such that when the extracted biomass is combusted, particulate matter emissions are very low. In certain embodiments, the particulate matter emissions are so low as to avoid the need for any additional cleaning device, and associated control system, in order to meet current emission regulations.

A seventh step may include treatment of the biomass extract to form a hydrolysate comprising fermentable hemicellulose sugars. In some embodiments, the biomass extract is hydrolyzed using dilute acidic conditions at temperatures between about 100° C. and 190° C., for example about 120° C., 130° C., 140° C., 150° C., 160° C., or 170° C., and preferably from 120° C. to 150° C.

The acid may be selected from sulfuric acid, sulfurous acid, or sulfur dioxide. Alternatively, or additionally, the acid may include formic acid, acetic acid, or oxalic acid from the cooking liquor or recycled from previous hydrolysis. Alternatively, hemicellulase enzymes may be used instead of acid hydrolysis. The lignin from this step may be separated and recovered, or recycled to increase the heating value of the pellets, or sent directly to the boiler.

An eighth step may include evaporation of hydrolysate to remove some or most of the volatile acids. The evaporation may include flashing or stripping to remove sulfur dioxide, if present, prior to removal of volatile acids. The evaporation step is preferably performed below the acetic acid dissociation pH of 4.8, and most preferably a pH selected from about 1 to about 2.5. The dissolved solids are concentrated, such as to about 10% to about 40% to optimize fermentable hemicellulose sugar concentration to a particular microorganism. Saccharomyces Cerevisiae fermentation can withstand dissolved solids concentrations of 30-50%, while Clostridia Acetobutylicum fermentation is viable at 10-20% concentrations only, for example.

In some embodiments, additional evaporation steps may be employed. These additional evaporation steps may be conducted at different conditions (e.g., temperature, pressure, and pH) relative to the first evaporation step.

In some embodiments, some or all of the organic acids evaporated may be recycled, as vapor or condensate, to the first step (cooking step) and/or third step (washing step) to remove assist in the removal of minerals from the biomass. This recycle of organic acids, such as acetic acid, may be optimized along with process conditions that may vary depending on the amount recycled, to improve the cooking and/or washing effectiveness.

Some embodiments of the invention enable processing of “agricultural residues,” which for present purposes is meant to include lignocellulosic biomass associated with food crops, annual grasses, energy crops, or other annually renewable feedstocks. Exemplary agricultural residues include, but are not limited to, corn stover, corn fiber, wheat straw, sugarcane bagasse, rice straw, oat straw, barley straw, miscanthus, energy cane, or combinations thereof. In certain embodiments, the agricultural residue is sugarcane bagasse.

Certain variations of the invention provide a process for producing biomass pellets and fermentable sugars from cellulosic biomass, the process comprising:

(a) providing a feedstock comprising cellulosic biomass;

(b) extracting the feedstock with steam and/or hot water under effective extraction conditions to produce an extract liquor containing hemicellulosic oligomers, dissolved lignin, and cellulose-rich solids;

(c) separating at least a portion of the cellulose-rich solids from the extract liquor, to produce dewatered cellulose-rich solids;

(d) hydrolyzing the dewatered cellulose-rich solids, thereby removing a portion of cellulose contained therein, to produce intermediate solids and a hydrolysate;

(e) drying and/or torrefying the intermediate solids to produce energy-dense biomass;

(f) pelletizing the energy-dense biomass to form biomass pellets;

(g) recovering fermentable sugars from the hydrolysate; and

(h) hydrolyzing the hemicellulosic oligomers contained in the extract liquor, under effective hydrolysis conditions, to produce hemicellulosic sugars.

Optionally, some or all of the hemicellulosic sugars are combined with the fermentable sugars derived from step (g), to form a combined biomass-sugars stream. In some embodiments, the hemicellulosic sugars are separately recovered from the fermentable sugars derived from step (g).

In some embodiments, the fermentable hemicellulose sugars are recovered from solution, in purified form. In some embodiments, the fermentable hemicellulose sugars are fermented to produce of biochemicals or biofuels such as (but by no means limited to) ethanol, 1-butanol, isobutanol, acetic acid, lactic acid, or any other fermentation products. A purified fermentation product may be produced by distilling the fermentation product, which will also generate a distillation bottoms stream containing residual solids. A bottoms evaporation stage may be used, to produce residual solids.

Following fermentation, residual solids (such as distillation bottoms) may be recovered, or burned in solid or slurry form, or recycled to be combined into the biomass pellets. Use of the fermentation residual solids may require further removal of minerals. Generally, any leftover solids may be used for burning as additional liquefied biomass, after concentration of the distillation bottoms.

Part or all of the residual solids may be co-combusted with the energy-dense biomass, if desired. Alternatively, or additionally, the process may include recovering the residual solids as a fermentation co-product in solid, liquid, or slurry form. The fermentation co-product may be used as a fertilizer or fertilizer component, since it will typically be rich in potassium, nitrogen, and/or phosphorous.

Optionally, the process may include co-combusting the recovered lignin with the energy-dense biomass, to produce power. The recovered lignin may be combined with the energy-dense biomass prior to combustion, or they may be co-fired as separate streams. When recovered lignin is combined with the energy-dense biomass for making pellets, the lignin can act as a pellet binder.

Part or all of the residual solids may be co-combusted with the energy-dense biomass, if desired. Alternatively, or additionally, the process may include recovering the residual solids as a fermentation co-product in solid, liquid, or slurry form. The fermentation co-product may be used as a fertilizer or fertilizer component, since it will typically be rich in potassium, nitrogen, and/or phosphorous.

In certain embodiments, the process further comprises combining, at a pH of about 4.8 to 10 or higher, a portion of the vaporized acetic acid with an alkali oxide, alkali hydroxide, alkali carbonate, and/or alkali bicarbonate, wherein the alkali is selected from the group consisting of potassium, sodium, magnesium, calcium, and combinations thereof, to convert the portion of the vaporized acetic acid to an alkaline acetate. The alkaline acetate may be recovered. If desired, purified acetic acid may be generated from the alkaline acetate, such as through electrolytic reduction to acetic acid.

In some variations, Green Power+® technology, commonly assigned with the assignee of this patent application, may be employed or modified as taught in one or more co-pending patent applications, U.S. patent application Ser. Nos. 12/474,267; 13/026,273; 13/026,280; 13/500,917; 61/612,453; 61/536,477; 61/612,451 and 61/624,880, which are hereby incorporated by reference herein in their entireties.

Some variations of this invention are premised on the realization that hydrothermal torrefaction may be incorporated into the process in several ways. As intended herein, “hydrotorrefaction” is equivalent to hydrothermal torrefaction,” “hydrothermal carbonization,” “wet torrefaction,” “wet carbonization,” “aqueous torrefaction,” “hydrothermal pretreatment,” and the like.

According to Yan et al., “Thermal pretreatment of lignocellulosic biomass,” Environmental Progress and Sustainable Energy 28(3) 435-440 (October 2009), wet torrefaction generally produces a solid with greater energy density than dry torrefaction, with the same mass yield. This disclosure incorporates by reference the public presentation Hoekman et al., “Hydrochar as a renewable fuel,” International Biomass Conference and Expo, April 16-19, Denver, Colo., for its teachings on the properties of solids that may be produced by hydrotorrefaction.

As opposed to dry torrefaction, which is conducted without a substantial amount of water added, if any, hydrotorrefaction is conducted with a substantial amount of hot water on intimate contact with the solids. The ratio of hot water to solids may vary widely, such as from about 1 to about 20 (ratio of water to biomass, by weight). In some embodiments, the water:biomass weight ratio is about 4, 5, 6, 8, or 10. Notably, because hydrotorrefaction requires water, it is not necessary to dry the solids following the previous step(s). Thus, wet biomass may be used directly. Additionally if there is any aqueous pretreatment or upstream aqueous processing prior to hydrotorrefaction, the water does not need to be removed until after the hydrotorrefaction has been performed.

In some embodiments, water for hydrotorrefaction is provided by introducing fermentation stillage or vinasse. The stillage or vinasse may be provided from an integrated process step that is co-located with the hydrotorrefaction, or the stillage or vinasse may be provided from an external source, such as a first-generation sugarcane ethanol plant. For example, sugars may be recovered from biomass (by extraction) and fermented. A fermentor stillage may then be provided to a hydrotorrefaction unit that converts the remaining solids (after extraction) to hydrochar. Alternatively, or additionally, stillage or vinasse may be utilized in the initial extraction and allowed to pass through the process to the hydrotorrefaction step. Components other than water in the stillage or vinasse may end up in the hydrochar, at least to some extent, in some embodiments.

It should also be recognized that there is a continuum from dry torrefaction (discussed previously) to wet torrefaction, and the present invention contemplates that any type of torrefaction may be utilized. Thus, in other embodiments as previously discussed, the water:biomass weight ratio is less than 1, such as about 0.8, about 0.5, about 0.2, about 0.1, about 0.05, about 0.02, about 0.01, or less, including substantially no water added or present.

In some embodiments, hydrotorrefaction is carried out at a temperature selected from about 150° C. to about 300° C., such as about 200° C. to about 275° C. Generally speaking, higher temperatures lead to higher energy density in the final solid product. Temperatures above about 300° C. are undesired for hydrotorrefaction because other chemical reactions will occur, such as pyrolysis, decomposition, or steam reforming of the solids.

In some embodiments, hydrotorrefaction is carried out at a residence time (or reaction time for a batch reactor) of about 2 minutes to about 60 minutes, such as about 5 minutes to about 15 minutes. Generally, lower reaction times will require higher reaction temperatures, and vice-versa.

In some embodiments, hydrotorrefaction is carried out at a pressure from about 100 psia to about 1,000 psia, such as from about 200 psia to about 800 psia. In some embodiments, the hydrotorrefaction pressure is not independently controlled but is determined by the equilibrium pressure at the selected temperature. The equilibrium pressure may be the static (closed system), saturated pressure within the reactor, for example. The hydrotorrefaction pressure may be controlled to a certain level if desired, such as by introducing an inert gas (e.g., N₂ or CO₂) into the reactor. In other embodiments, the hydrotorrefaction pressure is controlled or adjusted by withdrawing material from the hydrotorrefaction reactor, which may be periodic or continuous.

The hydrotorrefied solids from hydrotorrefaction can be characterized, in various embodiments, as solids with modest fuel densification (e.g., 10-50% increase in energy density), significant oxygen elimination, and increased friability. The hydrotorrefied solids are hydrophobic, or at least have an increased hydrophobicity relative to the starting material, so that the solids are suitable for storage and transportation. Also, the hydrotorrefied solids can be pelletized, with or without added binder. Components that are present may serve as a binder—including lignin, furans, resins, and humic acids.

There are many variations of process configurations that incorporate the principles of hydrotorrefaction coupled with the biomass-hydrolysis principles set forth herein. Without limitation, certain variations will now be described.

In a first variation, biomass is first extracted with steam or hot water, such as described previously, to produce a stream with hemicelluloses and extracted solids. The hemicelluloses may be separately processed to produce fermentable sugars, which then may be fermented to ethanol, 1-butanol, isobutanol, or other fermentation products. The extracted solids may be fed, optionally without any intermediate drying, to a hydrotorrefaction unit operated under suitable conditions, such as described above. The hydrotorrefied solids may then be pelletized, without any external binders, to produced pellets. The pellets may be used in combustion to produce heat and/or power.

In a second variation, biomass is first extracted with steam or hot water, such as described previously, to produce a stream with hemicelluloses and extracted solids. The hemicelluloses may be separately processed to produce fermentable sugars, which then may be fermented to ethanol, 1-butanol, isobutanol, or other fermentation products. The extracted solids may be fed, optionally without any intermediate drying, to a hydrotorrefaction unit operated under suitable conditions, such as described above. The hydrotorrefied solids may then be fed directly to a combustion unit to produce heat and/or power. Or, the hydrotorrefied solids may be co-fed with another solid fuel, such as coal, raw biomass, untorrefied biomass, dry-torrefied biomass, or lignin.

In a third variation, hydrotorrefied solids may be fed to a gasification unit to produce syngas (H₂ and CO). The syngas may then be used to produce heat and/or power. Alternatively, or additionally, the syngas may be used to produce fuels and chemicals, such as methanol or alcohols, hydrocarbons, olefins, or organic acids, by chemical catalysis or fermentation.

In a fourth variation, biomass is first extracted with steam or hot water, such as described previously, to produce a stream with hemicelluloses and extracted solids. The hemicelluloses may be separately processed to maximize production of furfural, 5-hydroxymethylfurfural, and/or levulinic acid (see further discussion below with respect to these sugar derivatives). The extracted solids may be fed, optionally without any intermediate drying, to a hydrotorrefaction unit operated under suitable conditions. The hydrotorrefied solids may then be pelletized, without any external binders, to produced pellets. The pellets may be used in combustion to produce heat and/or power.

In a fifth variation, biomass is first extracted with steam or hot water, such as described previously, to produce a stream with hemicelluloses and extracted solids. The hemicelluloses may be separately processed to maximize production of furfural, 5-hydroxymethylfurfural, and/or levulinic acid (see further discussion below with respect to these sugar derivatives). The extracted solids may be fed, optionally without any intermediate drying, to a hydrotorrefaction unit operated under suitable conditions, such as described above. The hydrotorrefied solids may then be fed directly to a combustion unit to produce heat and/or power. Or, the hydrotorrefied solids may be co-fed with another solid fuel, such as coal, raw biomass, untorrefied biomass, dry-torrefied biomass, or lignin.

In a sixth variation, extracted solids may be fed, optionally without any intermediate drying, to a hydrotorrefaction unit operated under conditions for, and configured for, producing two main products: hydrotorrefied solids (char), and levulinic acid. In some embodiments, the hydrotorrefaction unit is optimized to hydrolyze cellulose to C₆ sugars (e.g., glucose), followed by conversion of the C₆ to 5-hydroxymethylfurfural and then to levulinic acid, without allowing significant reactions of levulinic acid to carbonaceous solids. A separation unit may be in operable communication with the hydrotorrefaction unit, to separate levulinic acid as a distinct product.

In the sixth variation, the hydrotorrefied solids may then be pelletized, without any external binders, to produced pellets. The pellets may be used in combustion to produce heat and/or power. Or the hydrotorrefied solids may be fed directly to a combustion unit to produce heat and/or power, or co-combusted with another solid fuel.

In a seventh variation, biomass is first extracted with steam or hot water, such as described previously, to produce a stream with hemicelluloses and extracted solids. The extracted solids may be fed, optionally without any intermediate drying, to a hydrotorrefaction unit operated under conditions for, and configured for, producing two main products: hydrotorrefied solids (char), and levulinic acid. The hydrotorrefied solids may be pelletized. The hemicelluloses may be separately processed to produce fermentable sugars, which then may be fermented to ethanol, 1-butanol, isobutanol, or other fermentation products. Thus, for example, products from an integrated process may include energy-dense pellets, ethanol, and levulinic acid.

In an eighth variation, biomass is first extracted with steam or hot water, such as described previously, to produce a stream with hemicelluloses and extracted solids. The extracted solids may be fed, optionally without any intermediate drying, to a hydrotorrefaction unit operated under conditions for, and configured for, producing two main products: hydrotorrefied solids (char), and levulinic acid. The hydrotorrefied solids may be pelletized. The hemicelluloses may be separately processed to maximize production of furfural, 5-hydroxymethylfurfural, and/or levulinic acid. Thus, for example, products from an integrated process may include energy-dense pellets, furfural, and levulinic acid.

Other variations are premised on the recognition that not only can hydrotorrefaction be integrated downstream of biomass extraction/hydrolysis, but it can alternatively be integrated upstream, or in connection with, biomass extraction/hydrolysis.

In a ninth variation, biomass is fed to an extraction reactor operated under hydrotorrefaction conditions, with pressurized hot water. The liquid hot water may include process condensate from one or more downstream steps. Hemicelluloses are removed during hydrotorrefaction. In some embodiments, the hemicelluloses further react to sugar monomers. In certain embodiments, these sugar monomers are allowed to react to furfural, 5-HMF, and/or levulinic acid. The extracted solids undergo hydrotorrefaction, in the same unit or a separate unit. The hydrotorrefied solids may then be pelletized, without any external binders, to produced pellets. The pellets may be used in combustion to produce heat and/or power, for example. Or, the hydrotorrefied solids may be combusted or gasified directly.

In a tenth variation, biomass is fed to an extraction reactor operated under hydrotorrefaction conditions, with pressurized hot water. The liquid hot water may include process condensate from one or more downstream steps. Hemicelluloses are removed during hydrotorrefaction. In some embodiments, the hemicelluloses further react to sugar monomers. In certain embodiments, these sugar monomers are separately fermented to ethanol or another fermentation product. The extracted solids undergo hydrotorrefaction, in the same unit or a separate unit. The hydrotorrefied solids may then be pelletized, without any external binders, to produced pellets. The pellets may be used in combustion to produce heat and/or power, for example. Or, the hydrotorrefied solids may be combusted or gasified directly.

In the ninth and tenth variations, the product profile may be adjusted by varying the reaction conditions as well as the reactor configuration. For example, a continuous countercurrent reactor may be employed to optimize the release of hemicelluloses and conversion to sugars, while allowing the solid material to continue hydrotorrefaction. Many options and embodiments will be recognized. For example, in the ninth and tenth variations, the system may be configured to capture hemicellulosic sugars and/or furfural—as well as 5-HMF and/or levulinic acid derived from the cellulose in the solids—in addition to a final hydrotorrefied solids product for pelletizing or other use.

Some variations provide a process for producing energy-dense biomass and fermentable sugars from cellulosic biomass, the process comprising:

(a) providing a feedstock comprising cellulosic biomass;

(b) extracting the feedstock with steam and/or hot water under effective extraction conditions to produce an extract liquor containing hemicellulosic oligomers, dissolved lignin, and cellulose-rich solids, wherein at least some of the steam and/or hot water is derived from an internal or external source of fermentation vinasse;

(c) separating at least a portion of the hemicellulosic oligomers from the cellulose-rich solids, to produce intermediate solids;

(d) torrefying the intermediate solids to produce the energy-dense biomass;

(e) hydrolyzing the hemicellulosic oligomers into fermentable sugars; and

(f) recovering the fermentable sugars.

In some embodiments, step (d) utilizes hydrotorrefaction, i.e. torrefaction in the presence of water. In some embodiments, the extraction solution comprises pressurized hot water, which may be used also for hydrotorrefaction when employed. The extraction solution may further contains sulfur dioxide, sulfurous acid, sulfuric acid, or any combination thereof.

In some embodiments, step (c) includes washing the cellulose-rich solids using an aqueous wash solution, to produce a wash filtrate; and optionally combining at least some of the wash filtrate with the extract liquor. In certain embodiments, step (c) includes pressing the cellulose-rich solids to produce dewatered cellulose-rich solids and a press filtrate; and optionally combining at least some of the press filtrate with the extract liquor.

The process may further comprise refining or milling the intermediate cellulose-rich solids prior to step (d). The process may further comprise combusting the energy-dense biomass to produce power and/or heat. Alternatively, or additionally, the process may further comprise gasifying the energy-dense biomass to produce syngas.

The process further comprises, in some embodiments, a step of fermenting the fermentable sugars to a fermentation product. Optionally, the step of fermenting the fermentable sugars to a fermentation product generates fermentation vinasse that is recycled (and reused), at least in part, to step (b) (and/or to step (d) when hydrotorrefaction is employed). Alternatively, or additionally, the fermentation vinasse may be obtained from a separate, first-generation ethanol plant, directly or indirectly, for example.

In some embodiments, the process further comprises converting at least a portion of the hemicelluloses into furfural. At least a portion of the furfural may degrade to char, which may then be recovered with the energy-dense biomass. Similarly, the process may co-produce 5-hydroxymethylfurfural and/or levulinic acid from part of the cellulose-rich solids or the intermediate solids.

In some embodiments, the process further comprises pelletizing the intermediate solids and/or the energy-dense biomass, to produce biomass pellets.

Other variations of the invention provide a process for producing biomass pellets and fermentable sugars from cellulosic biomass, the process comprising:

(a) providing a feedstock comprising cellulosic biomass;

(b) extracting the feedstock with steam and/or hot water under effective extraction conditions to produce an extract liquor containing hemicellulosic oligomers, dissolved lignin, and cellulose-rich solids;

(c) separating at least a portion of the hemicellulosic oligomers from the cellulose-rich solids, to produce intermediate solids;

(d) hydrotorrefying the intermediate solids to produce the energy-dense biomass, wherein water for hydrotorrefaction is derived, at least in part, from an internal or external source of fermentation vinasse;

(e) pelletizing the energy-dense biomass to form the biomass pellets;

(f) hydrolyzing the hemicellulosic oligomers into fermentable sugars; and

(g) recovering the fermentable sugars.

In some embodiments, the extraction solution comprises pressurized hot water. The extraction solution may further contain sulfur dioxide, sulfurous acid, sulfuric acid, or any combination thereof.

The process may further comprise refining or milling the intermediate cellulose-rich solids prior to step (d).

The process may include combusting the biomass pellets to produce power and/or heat. In some embodiments, the biomass pellets have an energy content from about 8,500 Btu/lb to about 12,000 Btu/lb on a dry basis. The biomass pellets may also be gasified to produce syngas, which may be used for combined heat and power or for conversion to liquid fuels (e.g., Fischer-Tropsch synthesis to diesel fuels), for example.

In some embodiments, the process further comprises converting at least a portion of the hemicelluloses into furfural. Some or all of the furfural may degrades to char, and the char is optionally recovered with the energy-dense biomass. The process may also co-produce 5-hydroxymethylfurfural and/or levulinic acid from part of the cellulose-rich solids or the intermediate solids.

Some embodiments further comprise a step of fermenting the fermentable sugars to a fermentation product. This step may generate fermentation vinasse that is recycled, at least in part, to step (d) and/or to step (b). Alternatively, or additionally, fermentation vinasse is obtained from a separate, first-generation ethanol plant, and utilized in step (d) and/or step (b).

Certain variations provide a process for producing biomass pellets and fermentable sugars from cellulosic biomass, the process comprising:

(a) providing a feedstock comprising cellulosic biomass;

(b) extracting the feedstock with steam and/or hot water under effective extraction conditions to produce an extract liquor containing hemicellulosic oligomers, dissolved lignin, and cellulose-rich solids, wherein at least some of the steam and/or hot water is derived from an internal or external source of fermentation vinasse;

(c) separating at least a portion of the hemicellulosic oligomers from the cellulose-rich solids, to produce intermediate solids;

(d) hydrotorrefying the intermediate solids to produce the energy-dense biomass, wherein water for hydrotorrefaction is derived, at least in part, from the internal or external source of fermentation vinasse;

(e) pelletizing the energy-dense biomass to form the biomass pellets;

(f) hydrolyzing the hemicellulosic oligomers into fermentable sugars; and

(g) recovering the fermentable sugars.

Generally speaking, process conditions that may be adjusted to promote furfural, 5-hydromethylfurfural, and/or levulinic acid include, in one or more reaction steps, temperature, pH or acid concentration, reaction time, catalysts or other additives (e.g. FeSO₄), reactor flow patterns, and control of engagement between liquid and vapor phases. Conditions may be optimized specifically for furfural, or specifically for 5-hydromethylfurfural, or specifically for levulinic acid, or for any combination thereof.

The hemicelluloses that were initially extracted may then be processed to produce furfural and 5-hydroxymethylfurfural (HMF), in one or more steps. Some furfural and HMF may be produced during the initial extraction itself, under suitable conditions. In some embodiments, the hemicellulose-containing liquor is fed to a unit for production of furfural directly from C₅ monomers and oligomers and HMF directly from C₆ monomers and oligomers.

Thus in some embodiments, the hemicelluloses are first subject to a step to further hydrolyze the oligomers into monomers. This step may be performed with acids or enzymes. Depending on the feedstock, the hydrolyzed hemicelluloses will contain various quantities of C₅ sugars (e.g., xylose) and C₆ sugars (e.g., glucose).

In some embodiments, a reaction step is optimized to produce furfural. In some embodiments, a reaction step is optimized instead to produce HMF. In certain embodiments, a reaction step is configured to produce both furfural and HMF, which may be then separated or may be further processed together.

When it is desired to produce levulinic acid, the liquid may be further processed to convert at least some of the HMF into levulinic acid, with or without intermediate separation of furfural. In some embodiments, a reaction step is optimized to produce furfural, which is then recovered, followed by production of levulinic acid, which is separately recovered. In some embodiments, a single step is configured to produce both furfural and levulinic acid, which may be recovered together in a single liquid or may be separated from each other and then recovered. Conversion of HMF to levulinic acid also produces formic acid, which may be separately recovered, recycled, or purged.

In some embodiments, the furfural is further reacted, in the same reactor or in a downstream unit, to one or more acids such as succinic acid, maleic acid, fumaric acid, or humic acid. In some embodiments, conditions are selected to maximize conversion of furfural to succinic acid. In some embodiments, the furfural reacts to humic acid or char, in the hydrotorrefied solids or another solid stream.

In various embodiments, the process is configured to produce, in crude or purified form, one or more products selected from the group consisting of levulinic acid, furfural, 5-hydroxymethylfurfural, formic acid, succinic acid, maleic acid, fumaric acid, and acetic acid. Mixtures of any of the foregoing are possible. Any of these acids may be recycled in the process, such as to enhance the initial extraction of hemicelluloses or to enhance secondary hydrolysis of hemicellulose oligomers to monomers. Thus in some embodiments, acetic acid, formic acid, or other acids may be recovered and recycled.

Reaction conditions for producing furfural, HMF, and levulinic acid may vary widely (see, for example, U.S. Pat. Nos. 3,701,789 and 4,897,497 for some conditions that may be used). Temperatures may vary, for example, from about 120° C. to about 275° C., such as about 200° C. to about 230° C. Reaction times may vary from less than 1 minute to more than 1 hour, including about 1, 2, 3, 5, 10, 15, 20, 30, 45, and 60 minutes. The quantity of acid may vary widely, depending on other conditions, such as from about 0.1% to about 10% by weight, e.g. about 0.5%, about 1%, or about 2% acid. The acid may include sulfuric acid, sulfurous acid, sulfur dioxide, formic acid, levulinic acid, succinic acid, maleic acid, fumaric acid, acetic acid, or lignosulfonic acid, for example.

The residence times of the reactors may vary. There is an interplay of time and temperature, so that for a desired amount of hydrolysis or dehydration, higher temperatures may allow for lower reaction times, and vice versa. The residence time in a continuous reactor is the volume divided by the volumetric flow rate. The residence time in a batch reactor is the batch reaction time, following heating to reaction temperature.

The mode of operation for the reactor, and overall system, may be continuous, semi-continuous, batch, or any combination or variation of these. In some embodiments, the reactor is a continuous, countercurrent reactor in which solids and liquid flow substantially in opposite directions. The reactor may also be operated in batch but with simulated countercurrent flow.

When multiple stages are utilized, such as a first stage to produce or optimize furfural and HMF followed by a second stage to produce or optimize levulinic acid, the conditions of the second stage may be the same as in the first stage, or may be more or less severe. If furfural is removed, at least in part, a quantity of acid may also be removed (e.g. by evaporation) in which case it may be necessary to introduce an additional amount of acid to the second stage.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A process for producing energy-dense biomass and fermentable sugars from cellulosic biomass, said process comprising:
(a) providing a feedstock comprising cellulosic biomass;
(b) extracting said feedstock with steam and/or hot water under effective extraction conditions to produce an extract liquor containing hemicellulosic oligomers, dissolved lignin, and cellulose-rich solids, wherein at least some of said steam and/or hot water is derived from an internal or external source of fermentation vinasse;
(c) separating at least a portion of said hemicellulosic oligomers from said cellulose-rich solids, to produce intermediate solids;
(d) torrefying said intermediate solids to produce said energy-dense biomass;
(e) hydrolyzing said hemicellulosic oligomers into fermentable sugars; and
(f) recovering said fermentable sugars.

2. The process of claim 1, wherein step (d) utilizes hydrotorrefaction.

3. The process of claim 1, wherein said extraction solution comprises pressurized hot water.

4. The process of claim 1, wherein said extraction solution further contains sulfur dioxide, sulfurous acid, sulfuric acid, or any combination thereof.

5. The process of claim 1, wherein step (c) includes washing said cellulose-rich solids using an aqueous wash solution, to produce a wash filtrate; and optionally combining at least some of said wash filtrate with said extract liquor.

6. The process of claim 5, wherein step (c) further includes pressing said cellulose-rich solids to produce dewatered cellulose-rich solids and a press filtrate; and optionally combining at least some of said press filtrate with said extract liquor.

7. The process of claim 1, said process further comprising refining or milling said intermediate cellulose-rich solids prior to step (d).

8. The process of claim 1, said process further comprising combusting said energy-dense biomass to produce power and/or heat.

9. The process of claim 1, said process further comprising gasifying said energy-dense biomass to produce syngas.

10. The process of claim 1, said process further comprising a step of fermenting said fermentable sugars to a fermentation product.

11. The process of claim 10, wherein said step of fermenting said fermentable sugars to a fermentation product generates fermentation vinasse that is recycled, at least in part, to step (b).

12. The process of claim 1, wherein said fermentation vinasse is obtained from a separate, first-generation ethanol plant.

13. The process of claim 1, said process further comprising converting at least a portion of said hemicelluloses into furfural.

14. The process of claim 13, wherein at least a portion of said furfural degrades to char, and wherein said char is recovered with said energy-dense biomass.

15. The process of claim 1, wherein said process co-produces 5-hydroxymethylfurfural and/or levulinic acid from part of said cellulose-rich solids or said intermediate solids.

16. The process of claim 1, said process further comprising pelletizing said intermediate solids and/or said energy-dense biomass, to produce biomass pellets.

17. A process for producing biomass pellets and fermentable sugars from cellulosic biomass, said process comprising:
(a) providing a feedstock comprising cellulosic biomass;
(b) extracting said feedstock with steam and/or hot water under effective extraction conditions to produce an extract liquor containing hemicellulosic oligomers, dissolved lignin, and cellulose-rich solids;
(c) separating at least a portion of said hemicellulosic oligomers from said cellulose-rich solids, to produce intermediate solids;
(d) hydrotorrefying said intermediate solids to produce said energy-dense biomass, wherein water for hydrotorrefaction is derived, at least in part, from an internal or external source of fermentation vinasse;
(e) pelletizing said energy-dense biomass to form said biomass pellets;
(f) hydrolyzing said hemicellulosic oligomers into fermentable sugars; and
(g) recovering said fermentable sugars.

18. The process of claim 17, wherein said extraction solution comprises pressurized hot water.

19. The process of claim 17, wherein said extraction solution further contains sulfur dioxide, sulfurous acid, sulfuric acid, or any combination thereof.

20. The process of claim 17, said process further comprising refining or milling said intermediate cellulose-rich solids prior to step (d).

21. The process of claim 17, wherein said biomass pellets have an energy content from about 8,500 Btu/lb to about 12,000 Btu/lb on a dry basis.

22. The process of claim 17, said process further comprising combusting said biomass pellets to produce power and/or heat.

23. The process of claim 17, said process further comprising gasifying said biomass pellets to produce syngas.

24. The process of claim 17, said process further comprising converting at least a portion of said hemicelluloses into furfural.

25. The process of claim 24, wherein at least a portion of said furfural degrades to char, and wherein said char is recovered with said energy-dense biomass.

26. The process of claim 17, wherein said process co-produces 5-hydroxymethylfurfural and/or levulinic acid from part of said cellulose-rich solids or said intermediate solids.

27. The process of claim 17, said process further comprising a step of fermenting said fermentable sugars to a fermentation product.

28. The process of claim 27, wherein said step of fermenting said fermentable sugars to a fermentation product generates fermentation vinasse that is recycled, at least in part, to step (d).

29. The process of claim 17, wherein said fermentation vinasse is obtained from a separate, first-generation ethanol plant.

30. A process for producing biomass pellets and fermentable sugars from cellulosic biomass, said process comprising:
(a) providing a feedstock comprising cellulosic biomass;
(b) extracting said feedstock with steam and/or hot water under effective extraction conditions to produce an extract liquor containing hemicellulosic oligomers, dissolved lignin, and cellulose-rich solids, wherein at least some of said steam and/or hot water is derived from an internal or external source of fermentation vinasse;
(c) separating at least a portion of said hemicellulosic oligomers from said cellulose-rich solids, to produce intermediate solids;
(d) hydrotorrefying said intermediate solids to produce said energy-dense biomass, wherein water for hydrotorrefaction is derived, at least in part, from said internal or external source of fermentation vinasse;
(e) pelletizing said energy-dense biomass to form said biomass pellets;
(f) hydrolyzing said hemicellulosic oligomers into fermentable sugars; and
(g) recovering said fermentable sugars.
